(19) United States
(12) Patent Application Publication
(10) Pub. No.: US 2010/0030187 A1
(43) Pub. Date: Feb. 4, 2010

(54) Apparatus and Method for Controlling Headaches

(76) Inventor: Tian XIA, Chicago, IL (US)

Correspondence Address: BRINKS HOFER GILSON & LIONE, P.O. BOX 10395, CHICAGO, IL, 60610, US

(21) Appl. No.: 12/184,358

(22) Filed: Aug. 1, 2008

(51) Int. Cl. A61M 11/00

(52) U.S. Cl. 604/514; 128/200.23

(57) ABSTRACT

A user friendly apparatus and method provides effective pain control resulting from headaches and facial aches, which can be safely used by patients and medical personal alike. The economical apparatus can provide a controller equipped with an injector having a tubular section and nozzle, as well as a nostril-engaging introducer and an optional handle. In the user friendly method, the tubular section of the injector can be inserted through a nostril. The nozzle can then be positioned at an upward angle of inclination in proximity and downwardly and rearwardly of the sphenopalatine ganglion (SPG nerve). Thereafter, an anesthetic can be injected through the nozzle and sprayed upwardly, towards and about the SPG nerve to minimize pain and control a headache or facial ache.

### BACKGROUND OF THE INVENTION

This invention relates to headaches, and more particularly, to a user-friendly medical device and process to help control pain from headaches and facial aches.

Pain occurring from headaches and facial aches can be attributable to the following indications, symptoms, or particular circumstances. Sphenopalatine neuralgia, trigeminal neuralgia including glossopharyngeal neuralgia, migraine with or without aura; tension headaches; cluster headaches including chronic cluster headaches, paroxysmal hemicranias, superior laryngeal neuralgia, atypical facial pain, or herpes zoster opthalmicus.

It is very important and desirable to control the various forms of headaches and facial aches such as sphenopalatine neuralgia, trigeminal neuralgia including glossopharyngeal neuralgia, migraine with or without aura headaches, tension headaches, cluster headaches (including chronic cluster headaches), paroxysmal hemicranias, superior laryngeal neuralgia, atypical facial pain, and herpes zoster opthalmicus.

Migraine headache sufferers alone in the United States are estimated to be over 28 million people. Based on Medical Expenditure Panel Survey, total expenditures for these services averaged $4.3 billion per year in 2002 to 2003 with approximately 60% of the total for ambulatory visits and 40% for prescribed medicines. Another survey indicated that $13 billion in lost workdays and deceased productivity was due to migraines alone.

Available products for treating pain associated with headaches and facial aches are not as effective and as safe as desired. For example: NSAIDS brand medications including COX-2 brand medications can't be taken too much and/or too long for the danger of causing ulcer and maybe even heart attacks. These medications are also ineffective for a large portion of patients. Furthermore, narcotic medications are potentially addictive and therefore should be restricted. Use of Tryptan class medications such as Imitrex or Zomig brand medications are not only costly, but the potential toxicity can be so high that there is a restriction of doses a patient can take per day.

From an anatomical viewpoint, the sphenopalatine ganglion (SPG or SPG nerve) is the largest group of neurons outside the cranial cavity. The SPG is also sometimes referred to as the pterygopalatine nerve or ganglion. The SPG lies in the pterygopalatine fossa, which is approximately 1 cm wide and 2 cm high and resembles a vase on a lateral fluoroscopic view. The pterygopalatine fossa is bordered anteriorly by the posterior wall of the maxillary sinus, posteriorly by the medial plate of the pterygoid process, medially by the perpendicular plate of the palatine bone, and superiorly by the sphenoid sinus, and laterally it communicates with the infratemporal fossa.

The ganglion (SPG) within the fossa is located posterior to the middle turbinate of the nose and lies a few millimeters (1 mm to 5 mm) deep to the lateral nasal mucosa. The SPG has a complex neural center and has multiple connections. The SPG is suspended from the maxillary branch of trigeminal nerve at the pterygopalatine fossa via the pterygopalatine nerves and lies medial to the maxillary branch when viewed in the saggital plane. Posteriorly, the SPG is connected to the vidian nerve. The SPG itself has efferent branches and forms the superior posterior lateral nasal and pharyngeal nerves. Caudally, the ganglion (SPG) is in direct connection with the greater and lesser palatine nerves.

The SPG has sensory, motor and autonomic components. The sensory fibers arise from the maxillary nerve, pass through the SPG, and are distributed to the nasal membranes, the soft palate and some parts of the pharynx. A few motor nerves are also believed to be carried with the sensory trunks.

The autonomic innervations of the SPG are more complex. The sympathetic component begins with preganglionic sympathetic fibers originating in the upper thoracic spinal cord, forming the white ramie communicantes, coursing through the sympathetic ganglion, where the preganglionic fibers synapse with the postganglionic ones. The postganglionic fibers then join the carotid nerves before branching off and traveling through the deep petrosal and vidian nerves. The postganglionic sympathetic nerves continue their path through the SPG on their way to the lacrimal gland and nasal and palatine mucosa.

However, the overall function of the SPG is usually considered parasympathetic. The parasympathetic component of the SPG has its preganglionic origin in the superior salivatory nucleus then travel through a portion of the facial nerve (VII) before forming the greater petrosal nerve to form the vidian nerve, which ends in the SPG. Within the ganglion, the preganglionic fibers synapse with their postganglionic cells and continue on to the nasal mucosa, and one branch travels with the maxillary nerve to the lacrimal gland.

The SPGB procedure can be beneficial to some patients with pain, such as from: muscle spasm, vasospasm, neuralgia, reflex sympathetic dystrophy, chronic low back pain of multiple etiology, such as muscular, discogenic, arthritic, external cricoidynia, lower jaw toothache, glossodynia, earache in case of Eustachian tube and middle ear lesions, earache secondary to cancer of the larynx, pain from laryngeal tuberculosis, relief of spasm of the face and upper respiratory tract, syphilitic headache, malarial headache, cluster headache, ophthalmic migraine, dysmenorrheal, intercostal pain (neuralgia), gastric pain, nausea and diarrhea, myalgias of the neck muscles, sciatica, maxillary neuralgia, sensory facial neuralgia, pain in the upper teeth, tooth extraction, feeling of foreign body in the throat, persistent itching in the external ear canal, herpes zoster oticus, taste disturbances, atypical facial pain, tic douloureux, cervical arthritis, myofascial syndrome, peripheral neuropathy, post-herpetic neuralgia, fracture secondary to osteoporosis, lumbosacral strain, extremity arthritis and various other arthritic conditions. Further indication not related to pain control is the control of rage reaction and improvement of depression in the psychiatric patient.

While the SPG block can be effective in controlling chronic pain, it is performed by medical professionals, such as by using the pledget delivery method. In the pledget method, one or two cotton-tipped applicators are inserted into one of the two nostrils of the patient. There are two SPG, one on the left, one on the right. Using the middle turbinate as an anatomical landmark guideline, the two applicators are pushed upward until they contact the desired area in a blind approach. The success rate of the SPGB procedure is dependent on the experience of the physician. Lidocaine is applied on the cotton of the applicators and is then applied to the SPG area via the soaked cotton. The pledget method delivers an imprecise amount of medication to the area being treated and if in excess, can cause undesirable side effects such as throat irritation or systemic hypotension leading to shock and induce trauma to the nose. Furthermore, use of the pledget method can deliver less than an effective amount of medication, or miss the appropriate area to do this SPG block therefore resulting in failure of the desired results. These cotton-tipped applicators are usually kept inside the patient's nose for at least 30 minutes and often cause pain. It is desirably that the pledget method be performed by a medical doctor who is trained as a medical specialist in the areas such as: ear, nose and throat, rehabilitation medicine, neurology or anesthesiology in a hospital, doctor's office, clinic or other medical environments. There is therefore a need for a procedure to more effectively accomplish the SPG block procedure.

The sphenopalatine nerve block (SPGB) procedure is a non-invasive ganglion block for various chronic pains with no prolonged side effects and is done by physicians in a medical environment. A deposition of local anesthetics, such as lidocaine, onto the lateral nasal wall can be used which could penetrate the 1 mm to 5 mm layer of connective tissue and mucous membrane and exert pharmaceutical effects. This procedure appears to be somewhat beneficial for patients with various pain syndromes. In theory, blockade of SPG can inhibit the baseline tonic activity of sensory, sympathetic and parasympathetic function involving most parts of head and in so doing, it can control pain.

Clinically, SPG produce more parasympathetic activities; therefore SPGB produced more parasympathetic tone inhibition. Since most headaches, such as migraine headaches have vasodilatation roots in their etiology and a blockade of parasympathetic nerve function creates vasoconstriction, SPGB can be especially effective in relieving and controlling headaches. However, due to lack of uniform results and difficulty in performing the SPGB procedure, the SPGB procedure has not been utilized as much as it could be in actual medical practice.

Anatomically, there are many potentially severe side-effects associated with SPGB procedures. The roofapex of our nasal cavity is called cribriform plate of ethmoid bone. Olfactory nerve penetrates this thin bone through many foramina to reach the nasal cavity. Physical damage to this thin bone either with a cue tip or with a tube could cause severe brain damage and/or death. Spraying local anesthetics on to these areas can potentially result in total spinal block and then death of the patient if not resuscitated immediately. Furthermore, the cave of the SPG resides and is very hidden in the lateral nasal cavity. Many trained doctors have problems actually allocating and perform the SPGB procedure effectively.

Over the years, various techniques have been suggested for SPGB procedures. Dr. Jordon Yee from New York had applied U.S. Pat. No. 4,886,493 for medical applicator process for patients to perform a SPGB procedure. Dr. Jordon Yee's method has had very limited success and has not been widely utilized by the medical community. The SPGB procedure can not readily be performed safely with the Dr. Yee or other devices for the possibilities of total spinal block when sprayed on the apex of nostril. Furthermore, the SPGB procedure can not be performed effectively with Astra Zeneca device because SPG hides on the lateral wall of the nostril, and simply squeezing medication into the nose will not contact the SPG.

Headaches are a common symptom of numerous diseases and disorders including, but not limited to, migraine, muscle tension, systemic or intracranial infection, intracranial tumor, head injuries, severe hypertension, cerebral hypoxia, certain diseases of the eyes, nose, throat, teeth, and ears, and head pain.

Infrequent headaches can often be determined to result from causes attributable to a particular experience of a patient, such as fatigue, fever, alcohol ingestion, muscle contraction, tension, or the like. The cause of persistent or recurrent headaches is often difficult to determine. Persistent or recurrent headaches can include, but are not limited to, muscular headaches, such as tension or muscle contraction headaches, and neurovascular headaches, such as migraines and cluster headaches.

Cerebral neurovascular disorders are characterized by one or more disturbances in the normal functioning of at least one component of the cerebral vascular or nervous system in a human. Cerebral neurovascular disorders include, for example, migraine, cluster headaches, other headaches of neurovascular etiology, tinnitus, and cerebrovascular spasm. Patients afflicted with a cerebral neurovascular disorders can experience a single episode of the disorder, recurrent episodes, persistent episodes, or some combination of these patterns. An individual episode is designated an acute cerebral neurovascular disorder.

A migraine (migraine headache) is a disorder characterized by persistent headache, which can be severe, and can be associated with visual and gastrointestinal disturbances, and which can also be recurrent. In certain cases, visual changes (designated “aura” by some practitioners) or other symptoms precede the onset of a migraine. Such prodromal symptoms can be due to intracranial vasoconstriction. The precise etiology of migraine is unknown. Some reports suggest that a genetically transmitted functional disturbance of intra- and extra cranial circulation can be involved. Regional alterations in cerebral blood flow attributable to intracranial arterial vasodilatation are known to accompany headache associated with migraine. Some reports have attributed head pain associated with a migraine to substances released as a result of or associated with dilation of scalp arteries during an acute migraine episode.

Prodromal symptoms of an acute migraine episode can include, but are not limited to, depression, irritability, restlessness, anorexia, scintillating scotomas, visual changes such as perception of stars or zigzag lines, paresthesias, and hemiparesis. These prodromal symptoms can disappear shortly before the migraine is manifested, or can persist until or after the onset of the migraine.

Head pain associated with migraine can be unilateral or generalized. Nausea, vomiting, and photophobia often accompany migraines. Symptoms generally follow a pattern in an individual patient, except that unilateral head pain can not always be on the same side. Patients afflicted with migraine can experience migraines with a frequency between daily and only once in several months. An untreated acute migraine episode can endure for a long period, often hours or days.

Various surgical procedures and psychological counseling have been recommended to help alleviate the frequency of recurrence of migraines, such as surgery, participation of the patient in biofeedback procedures, administration of methysergide, propanolol, or a calcium channel blocker such as verapamil, or the administration of an ergotamine preparation such as dihydroergotamine, or a serotonin receptor agonist such as sumatriptan. Some procedures and psychological counseling can offer benefit to certain patients, but are not generally useful for alleviating the pain associated with an acute migraine.

Patients with acute migraine episode have been treated with various prescription and over-the-counter medication, such as: aspirin, codeine, a serotonin agonist such as sumatriptan, ergot, ergotamine, caffeine, a narcotic, butorphanol tartrate, meperidine, or a combination thereof. Administration of a combination of the preceding has not generally provided satisfactory or sustained relief from pain or other symptoms associated with an acute migraine episode in many patients. Furthermore, numerous side effects have been reported to accompany administration of these medications, including: dizziness, nausea, somnolence, fatigue, chest pain, cardiac infarction, hypertension, hypertensive crisis, chest-, face-, and neck-hyperemia, gastrointestinal upset, sedation, or drug dependence. Moreover, certain of these compounds are contraindicated for numerous patients, such as pregnant women, nursing women, patients using monoamine oxidase inhibitors, patients having a history of ischemic heart disease, ulcer, gastritis, kidney disease, liver disease, and other diseases. Other migraine treatments involve administration of a pharmaceutically active agent which interacts with a serotonin receptor on cerebral arterial surfaces.

A cluster headache comprises a headache which is characterized by recurrent episodes of unilateral excruciating pain, usually occurring on the same side of the head of a patient. Cluster headaches are typically oculofrontal or oculotemporal, with occasional radiation to the upper jaw, and are described as being of a boring, non-throbbing nature. Associated with the head pain are one or more autonomic accompaniments, such as: conjunctiva injection, nasal congestion, lacrimation, rhino rhea, body temperature elevation, vasodilatation on the same side as that on which the pain is experienced, and edema beneath the eye. A cluster headache is usually of short duration, persisting for between 15 and 90 minutes, and tends to occur in clusters, typically a few times a day for a period of 6 to 12 weeks. Months or years can pass between the clusters of headaches. Because headaches which appear to be identical to spontaneous cluster headaches can be induced by subcutaneous injection of histamine diphosphate, cluster headaches are also known as histamine headaches. Headaches having sensory similarity to cluster headaches can also be induced by administration of nitroglycerin to a human patient, for example by sublingual administration of 0.4 milligrams of nitroglycerin.

Treatments of patients with cluster headaches include the administration of: methysergide, a vasoconstrictor, corticosteroid, oxygen, indomethacin, intranasal administration of cocaine, which is a toxic shorter-acting local anesthetic with pronounced central effects and a vasoconstrictor, or lidocaine, which is also a shorter-acting local anesthetic. Shorter-acting local anesthetics can be effective to abort pain associated with a single individual headache episode that is only one of several headache episodes comprising a cluster headache, sometimes referred to as a cluster period. Large amounts of drug and repeated dosings are often required to achieve these results. Ropivacaine is an amino amide local anesthetic that is commercially available as the S(levo)-enantiomer. Ropivacaine allows differential nerve block and exhibits intermediate distribution and clearance, as well as has inherent vasoactive properties. The duration of the anesthetic effect at a given site of administration of a local anesthetic is dependent upon the total dose, the concentration, and the identity of the local anesthetic administered to the patient, the rate of systemic absorption, and often the presence or absence of a vasoconstriction or other agent in the anesthetic composition.

Muscular headaches are very common in the adult population. It is estimated that between about 3% and about 5% of patients who experience a muscular headache are afflicted with chronic muscular headaches. Muscular headache can occur more than 15 days per month for a period of at least about 6 months. Analgesic addiction is a recognized problem in the treatment of patients afflicted with chronic muscular headaches. Muscular headaches can be acute, such as is the case for typical episodic tension headaches, which are related to contraction of muscles of the head and neck. Sustained contractions of skeletal muscles of the head, neck, face, and shoulders are associated with concurrent local chemical changes within skeletal muscle, and can give rise to pain. The pain can be localized or it can be referred, so that the pain is perceived at a body location different than the location of muscle contraction. Muscle contraction headaches can also be chronic and associated with depression or with one or more other psychological problems. Muscle contraction headaches can also be associated with anatomic factors such as: cervical arthritis, temporomandibular joint disorders, irritating lesions, pressure and mechanical stress, eye strain, or emotional stress or disorders.

Muscular headaches, including muscle contraction headaches and tension headaches, are recognized as the most common category of recurring head pain. In contrast to migraines, muscular headaches are usually bilateral, often with occipital nuchal, temporal, or frontal predominance or with diffuse extension over the top of the cranium. The pain can be located in the back of the head and neck as well. Contrary to the pain from a migraine, the pain associated with a muscular headache is usually described as squeezing and vise-like in nature. Nausea, photophobia, and phonophobia are not generally associated with muscular headache episodes. The onset of a muscular headache episode is more gradual than the onset of a migraine or cluster headache episode, and muscular headache episodes are not generally associated with auras or prodromal symptoms. The onset of muscular headache episodes does not appear to be associated with physical activity by the patient. Once established, a muscular headache episode can persist, perhaps with minimal fluctuations in intensity, for weeks or months.

Although patients afflicted with migraine can be awakened from sleep, patients afflicted with a chronic muscular headache generally sleep undisturbed and perceive development or intensification of the headache soon after waking. About a third of patients afflicted with a muscular headache exhibit symptoms of depression. Migraine headaches can be complicated by tension headaches which persist and arouse fears of mass lesions, thereby leading to the performance of unnecessary diagnostic workups in many patients.

Muscular headaches are, in part, related to sustained contraction of the skeletal muscles of the scalp, face, neck, and shoulders. Sustained muscle contraction is related to local pathology, central influences, and multisystem modulation, and involves gamma efferent neuronal muscle spindle activation. Related monosynaptic conduction through the ventral horn augments both efferent neuronal discharge and muscle contraction. A cycle of pain, muscle spasm, local chemical changes, neural excitability, skeletal muscle blood vessel compression or spasm, and anxiety can occur with muscular headaches. Persistent headaches can cause sustained cranial muscle contraction, resulting in pain is typified by an aching sensation, rather than by the characteristic squeezing pain associated with muscular headaches. It is suspected that muscular headaches are not caused solely by sustained cranial muscle contraction.

Generally, the pain associated with a muscular headache episode is mild to moderate in severity, although the pain can become severe in many patients. Relaxation, massage, and common analgesic medications, such as aspirin and acetaminophen, can sometimes be effective to alleviate mild muscular headache pain. Codeine or other narcotic preparations, tranquilizers, and antidepressants are sometimes administered to patients experiencing more severe muscular headache pain. Unfortunately, many of these patients develop physical dependence on these agents and must be followed closely because of a significant incidence of addiction.

The musculature of the head, neck, jaw, or upper back is tense and tender in many or most patients afflicted with a muscular headache, and one or more trigger points, or muscle knots, are often present. Cervical spine arthritis and temporomandibular joint disorders can contribute to the development of a muscular headache.

Treatments which have been recommended for muscular headaches include: reassurance and psychological support, massage of the head and neck, application of hot and cold packs, transcutaneous electrical neural stimulation, physical support, such as the use of orthopedic pillows, aspirin, acetaminophen compounds, non-steroidal anti-inflammatory drugs, tricyclic antidepressants, narcotic analgesics, oral muscle relaxants, with or without tranquilizers, muscle relaxants, and other analgesic compounds. These treatments can sometimes be effective for alleviating mild- to moderate-intensity acute muscular headaches. Many patients who initially respond to one or more of these therapies become less responsive to these treatments, possibly because the patients develop a tolerance to the preceding medications, or because the disease process progresses or increases. Additionally, symptoms can be influenced by psychological factors which can remain constant or worsen. The side effects which accompany administration of known medications are significant and can become more severe.

Numerous pharmaceutically active agents can be useful when delivered systemically to a patient. Systemic delivery of such agents can sometimes be affected by oral administration of a composition comprising the agent. However, many pharmaceutically active agents are degraded by, or otherwise react with acids, proteins, or other agents located in, the human gastrointestinal tract or the human liver or circulatory system, with the result that the agent loses its pharmaceutical usefulness. For this reason, many pharmaceutically active agents can not practically be administered by an oral route to achieve systemic delivery of the agent. Furthermore, gastrointestinal absorption of an orally administered medication can be impaired in a distressed patient, such as a patient experiencing a migraine or any severe headache. Pharmaceutically active agents intended for systemic delivery to a patient can be administered intravenously. However, such methods can cause discomfort to the patient and often can be performed only in conjunction with frequent or continuous supervision of a medical professional.

Topically administering compositions to human tissue for systemic delivery of a pharmaceutically active agent include: the use of transdermal or transmucosal pastes, creams, liquids, solids or semisolid. Systemic delivery of a pharmaceutically active agent effected by topical administration methods are limited by the ability of the agent to diffuse through the tissue to which the composition is applied to reach blood vessels where the agent is absorbed for systemic delivery.

It is, therefore, desirable to provide an improved apparatus and method for controlling headaches, which overcomes many, if not all of the preceding disadvantages.

### BRIEF SUMMARY OF THE INVENTION

An improved apparatus and method is providing for controlling headaches and facial aches which is effective, easy to use and safe. Advantageously, the user friendly controller (apparatus) and method is economical and provides effective pain control for many patients suffering from sphenopalatine neuralgia, trigeminal neuralgia, gloss pharyngeal neuralgia, migraine headaches with or without aura, tension headaches, cluster headaches including chronic cluster headaches, paroxysmal hemicranias, superior laryngeal neuralgia, atypical facial pain, and herpes zoster opthalmicus.

Advantageously, while the novel apparatus and method can be readily used on a patient by an anesthesiologist and other physicians, nurses, or medical technicians, the patients themselves can safely and effectively use the special self-help apparatus and method independently without the presence of a doctor or other medical specialist. Desirably, the discomfort and cost of treating headaches are significantly improved and decreased by the attractive apparatus and method.

The special headache controlling apparatus provides a controller, device and medical equipment which can comprise a slidable and controlling moveable injector with a passageway for passing, delivering and injecting an anesthetic. The injector can comprise an elongated tubular section, an container-supporting semi-rigid stem that extends outwardly, upwardly or at an angle of inclination from one end of the tubular section, and a nozzle that can extend forwardly at an upward angle of inclination from the other end of the tubular section. The nozzle can have a tip with an array of apertures (holes) to spray an anesthetic toward a sphenopalatine ganglion (SPG nerve). A container of anesthetic can be secured to the stem to inject the anesthetic in the container through the stem and tubular section outwardly through the apertures of the nozzle. The special headache controlling apparatus (controller) can also comprise a nostril-engaging introducer and an optional handle.

In use, the handle can be pushed towards the patient's face until the introducer snugly and comfortably engages and fits within the nostril of the patient to preferably lift the flat tip of the nose to point upwardly, then anteriorly, and thereafter the stem and nozzle can be pushed toward the patient's nose to slide the tubular section and nozzle rearwardly until the nozzle is located medially, posteriorly and inferiorly to the SPG so as to be positioned in proximity of and rearwardly and downwardly of the SPG nerve so that the anesthetic in the container can be dispensed to inject a spray of the anesthetic through the apertures of the nozzle about the SPG nerve so as to substantially control, decrease and minimize pain from a headache or facial ache.

The user friendly method provides a special process, procedure and technique to effectively control headaches and facial aches. In the user friendly method, the tubular section of the injector can be inserted through a nostril. The nozzle can then be positioned at an upward angle of inclination in proximity and downwardly and rearwardly of the SPG nerve. Thereafter, an anesthetic can be injected and sprayed through apertures of the nozzle upwardly, laterally and anteriorly towards and about the SPG nerve to minimize pain and control a headache or facial ache. When the desired, appropriate, and/or proportioned amount of local anesthetic is sprayed onto the SPG nerve, it can result in immediate and prolonged vasoconstriction of the blood vessels in the ipsilateral head or brain and, thereafter, result in effective control of the patient's headache.

In the preferred apparatus and method, part of the apparatus (controller) can be inserted into a patient's nostril. A special apparatus is designed for each nostril, so that there are two complementary controllers, one for the right nostril and one for the left nostril. The introducer of the controller can be aimed to access the nostril and provide a horizontal pathway that can be parallel to the floor of the nose into the nostril to a position immediate medial to the inferior turbinate. The introducer can provide an extended pathway of about 1.5 cm to about 2 cm into the nostril. Once the introducer is placed firmly against the nose, the tip of the nose is no longer pointing inferiorly, but rather anteriorly. The tubular section of the injector, which is sometimes referred to as a tube or Cobra tube, can then be pushed partially or all the way into the back of the nostril. In order to ensure the slightly curved nature of the interior anatomy of the nose, the tunnel which the tubular section lies can be curved slightly to the ipsilateral nostril for about 5 degrees to about 20 degrees. After the tubular section is extended into the nose, the nozzle providing a head or mouth of the injector is designed, contoured and constructed to point lateral, anterior and superior from about 45 degrees to about 60 degrees. This design accommodates the anatomy of the patient where the SPG lies in the lateral cave right posterior to the middle turbinate. Once the tubular section is in position, the local anesthetic mixture in the container is dispensed in an amount, such as about 0.1 cc to 1 cc of liquid and then delivered accurately onto the SPG to exert the effect of a SPG nerve block.

A more detailed explanation of the invention is provided in the following detailed descriptions, examples and appended claims taken in conjunction with the accompanying drawings.

### BRIEF DESCRIPTION OF THE DRAWINGS

FIG. 1 is a cross-sectional side view an apparatus for controlling headaches and facial aches before being inserted into a patient's nostril in accordance with principles of the present invention.

FIG. 2 is a cross-sectional top plan view of the apparatus taken substantially along lines 2-2 of FIG. 1.

FIG. 3 is a cross-sectional side view the apparatus after the introducer and have been inserted into a patient's nostril in accordance with principles of the present invention.

FIG. 4 is a cross-sectional side view the apparatus with the nozzle positioned in a spraying position in proximity to and downwardly and rearwardly of the SPG nerve in accordance with principles of the present invention.

### DETAILED DESCRIPTION OF THE INVENTION

The following is a detailed description and explanation of the preferred embodiments of the invention and best modes for practicing the invention.

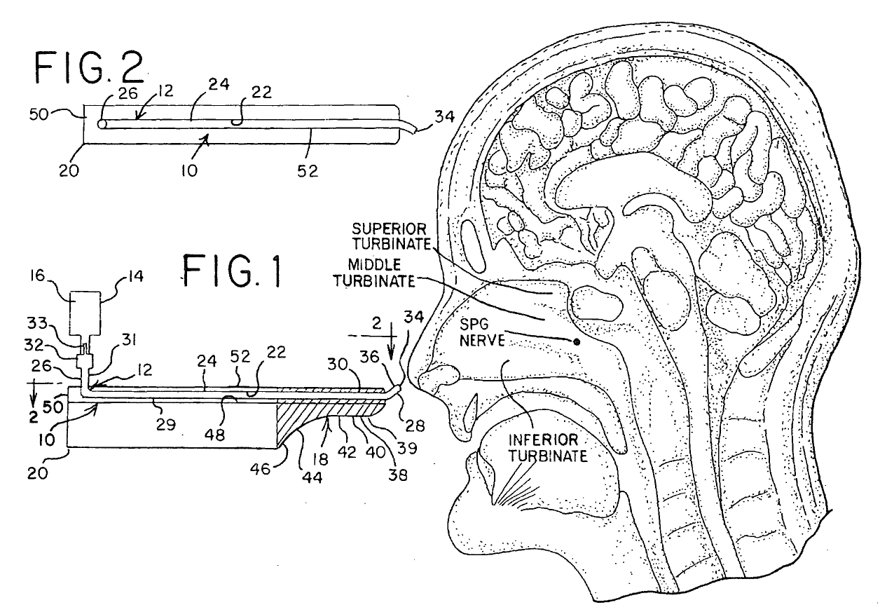

A special user-friendly headache controlling disposable apparatus 10 (FIGS. 1-4) provides a controller, device and medical equipment which can comprise a slidable and controlling moveable injector 12, a container 14 of anesthetic 16; a nostril-engaging introducer 18, and an optional handle 20. The injector has a passageway 22 for passing, delivering and injecting the anesthetic from the container. The injector can be molded of flexible plastic. While plastic is preferred, other materials can be used. The injector can comprise an elongated tubular section 24 (tube or Cobra tube), a container-supporting semi-rigid stem 26, and a nozzle 28. The tubular section can have a posterior portion 29 comprising a rearward end and an anterior portion 30 comprising a front end. The tubular section can have an outside diameter of about 5 mm and an interior diameter providing the passage of about 2 mm. While these diameters are preferred, if desired other diameters can be used. The stem can provide a superior pedestal with a lower stem section 31 which can have a similar outside diameter as the elongated tubular section and which can extend outwardly, upwardly or at an angle of inclination, from the posterior portion of the tube and can have an enlarged diameter upper stem section 32 to receive the outlet 33 of the container. The stem section can be vertical or positioned outwardly or at an angle of inclination. The nozzle can extend forwardly at an upward angle of inclination from the anterior portion of the tubular section. The nozzle can have a tip 34 with an array of apertures 36 that provide a series, pattern, cluster, array, or group of holes to spray the anesthetic toward a sphenopalatine ganglion (SPG nerve) of the patient. The nozzle can extends in a lateral, anterior and superior direction at an angle of inclination ranging from about 45 degrees to about 60 degrees. The nozzle can have a length ranging from 2 mm to 5 mm. The injector can be a left nostril-engaging injector or a right nostril-engaging injector. The left nostril-engaging injector has a different nozzle that is generally complementary in shape to the right nostril-engaging injector.

The container 14 (FIGS. 1, 3 and 4) can partially or fully contain an anesthetic 16. The container can be positioned and operatively connected, mounted or otherwise secured to the outer end of the stem. The container communicates with the passageway in the injector for injecting the anesthetic through the passageway of the stem and tubular section through the apertures of the nozzle. The container can be made of metal or plastic and can comprise: a squeezable container, a canister, a pressurized container, an aerosol can, or a syringe.

The nostril-engaging introducer 18 (FIGS. 1-4) can have a narrow anterior section 38 with a rounded convex arcuate anterior portion 39 and an underside 40 with a general planar flat surface 42 and a concave posterior section 44 having a larger cross-sectional than the narrow anterior section. The concave posterior section can have a contour 46 with a shape that is generally complementary and conforms to the interior of a patient's nostril for insertion in the nostril. The introducer can have a tube-receiving passageway 48 that extends therethrough to slidably receive the tubular section of the injector. The tube-receiving passageway can have a diameter of about 6 mm to about 7 mm. The anterior section 38 of the introducer can comprises a nostril-engaging tip that extends from about 1 cm to about 3 cm. The concave posterior section of the introducer can extend from about 2 cm to about 3 cm. The introducer can be elastomeric and/or resilient plastic or rubber. If desired, other materials can be used. The said introducer can be left nostril-engaging introducer or a right nostril-engaging introducer. The left nostril-engaging introducer has a different and generally complementary contour to the right nostril-engaging introducer.

The handle 20 (FIGS. 1-4) can comprise a manually grippable (graspable) handle which is securely connected to a back portion of the introducer. The handle can have and define an upwardly facing channel 50 providing a track 52 which communicates with the tube-receiving passageway of the introducer to slidably receive the elongated tubular section of the injector. The track can have a depth or width of about 6 mm to about 7 mm. The handle can be made of plastic, but if desired, other materials can be used.

The local anesthetic (medication) in the container can be a pressured aerosolized mixture of the following: benzocaine; tetracaine; and ropivacaine. Preferably, the anesthetic has a composition which comprises by weight based on the total weight of the anesthetic: about 14% benzocaine; about 2% tetracaine; and about 1% ropivacaine. The composition of the anesthetic can also comprise: preservatives, water of saline or water based soluble. The dosage or amount of anesthetic that can be used can range from 0.1 cc to 1.0 cc, preferable about 0.5 cc. The mixture (anesthetic) of benzocaine; tetracaine; and ropivacaine, can ensure the fast onset action of the SPGB as well as prolonged effect and thereby decreases reduce the need for repeat procedures and possibilities for potential dose related complications and side effects. The benzocaine onset time is about 30 seconds, lasting 0.5 to 1 hour, and has a toxic dose of more than 200 mg. By using this medication, there can be an almost immediate onset of pain relief, thereby decreasing the need to re-spray. Benzocaine is also very effective when used topically. Clinically, benzocaine may increase the absorption of other local anesthetics when mixed. Ropivacaine can have a slow onset, however it last anywhere from 2 to 6 hours. The toxic dose of ropivacaine is about 175 mg. By using this medication, patients will have an extended nerve block and pain relief effect. Tetracaine can also have a fast onset lasting about 0.5 to 1 hour. Tetracaine is also a very intense local anesthetic. When tetracaine is combined with ropivacaine, the pain relief effect lasts much longer than 6 hours.

The user friendly method provides a special process, procedure and technique for effectively controlling headaches and facial aches. In the user friendly method, the tubular section of the injector can be inserted through a nostril. The nozzle can then be positioned at an upward angle of inclination in proximity and downwardly and rearwardly of the SPG nerve. Thereafter, an anesthetic can be injected and spayed through apertures of the nozzle upwardly, laterally and anteriorly towards and about the SPG nerve to minimize pain and control a headache or facial ache.

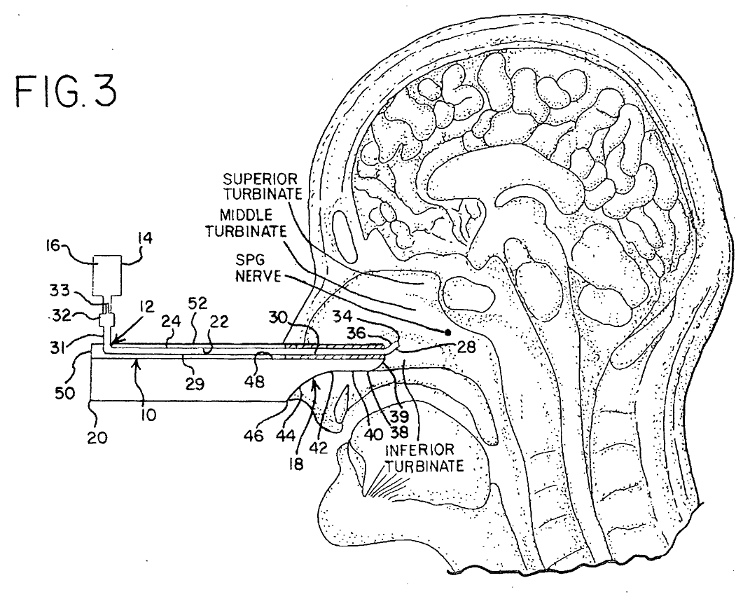

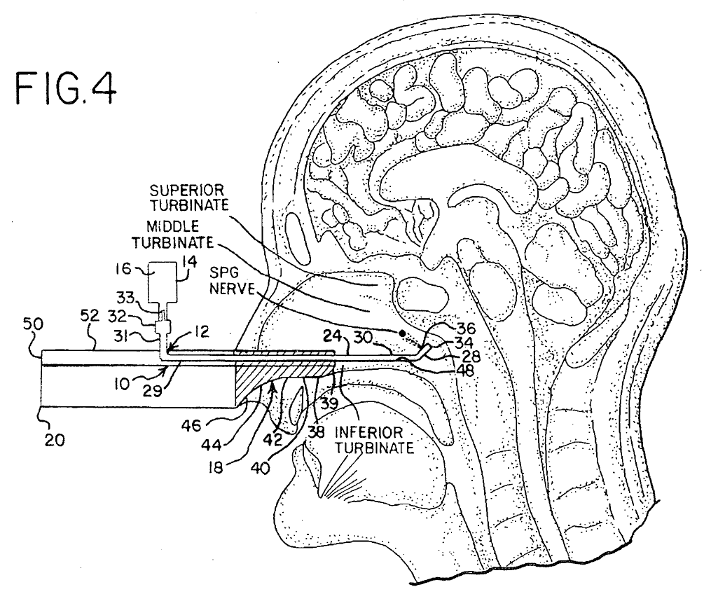

In a preferred method, the handle is pushed towards the patient's face until the introducer snugly and comfortably engages and fits within the nostril of the patient to lift the flat tip of the nose anteriorly or upwardly as shown in FIG. 3. Thereafter, the stem and nozzle are pushed from the storage position, which can be held by an option safety abutment stop, toward the patient's nose to slide the elongated tubular section and nozzle rearwardly in the injection position until the nozzle is located medially, posteriorly and inferiorly to the SPG so as to be positioned in proximity of and rearwardly and downwardly of the SPG nerve as shown in FIG. 4. If desired, the handle can be held with one hand and the stem or container can be pushed with the other hand. Afterwards, the anesthetic in the container can be dispensed to inject a spray of the anesthetic through the apertures of the nozzle about the SPG nerve to substantially control, decrease and minimize pain from a headache or facial ache. When the desired, appropriate, and/or proportioned amount of local anesthetic is sprayed onto the SPG nerve, it will result in immediate and prolonged vasoconstriction of the blood vessels in the ipsilateral head/brain and, thereafter, result in effective control of the patient's headache.

More preferably, the method can include pushing or placing the introducer snugly and comfortably within a nostril to lift the tip of the nose before positioning the nozzle in proximity to the SPG nerve; sliding the tubular section of injector through passageway in the introducer, and/or sliding the tubular section of the introducer on a track of the handle. This step can ensure the proper and safe passage of the tubular section rearwardly to reach in proximity to the SPG.

As previously indicated, in the preferred apparatus and method, part of the apparatus (controller) can be inserted into a patient's nostril. A special apparatus is designed for each nostril, so that there are two complementary controllers, one for the right nostril and one for the left nostril. The introducer of the controller can be aimed to access the nostril and provide a horizontal pathway that can be parallel to the floor of the nose into the nostril to a position immediate medial to the inferior turbinate. The introducer can provide an extended pathway of about 1.5 cm to about 2 cm into the nostril. Once the introducer is placed firmly against the nose, the nostril is no longer pointing inferiorly, but rather anteriorly. The tubular section of the injector, which is sometimes referred to as a tube or Cobra tube, can then be pushed partially or all the way into the back of the nostril. In order to ensure the curved nature of the interior anatomy of the nose, the tunnel which the tubular section lies can be curved slightly to the ipsilateral nostril for about 5 to 20 degrees. After the tubular section is extended into the nose, the nozzle providing a head or mouth of the injector is designed, contoured and constructed to point lateral, anterior and superior from about 45 degrees to about 60 degrees. This design accommodates the anatomy of the patient where the SPG lies in the lateral cave right posterior to the middle turbinate. Once the tubular section is in position, the local anesthetic mixture in the container is dispensed in an amount, such as about 0.1 cc to 1 cc of liquid and then delivered accurately onto the SPG to exert the effect of a SPG nerve block. Patients can use this inventive technique twice a hour or as needed. The novel apparatus and method can provide effective results on most patients over 15 years of age for 95% of the population and does not usually depend on the height, weight, sex or race of the patient.

Advantageously, surprisingly and unexpectedly, the inventive apparatus and method can control the pain and headache or facial ache in about 30 seconds to about 60 seconds after the injection of the anesthetic and for about 4 hours to about 24 hours after injection of the anesthetic.

### EXAMPLES 1-30

The subject apparatus and/or method (techniques) were performed in about 30 patients who suffer chronic headaches such as migraine and tension headaches. The preceding achieved surprisingly and unexpectedly results. The inventive technique resulted in 90% pain relief in 100% of the patients with 100% of each SPG nerve block. The onset of pain relief from the technique for the patients ranged from about 30 seconds to 60 seconds. The duration of pain relief from the technique for the patients ranged from about 4 to 24 hours. Each SPG nerve block was performed with only 0.5 cc or less of the local anesthetic mixture (composition) of benzocaine, tetracaine and ropivacaine as previously described. In at least 10 of the patients, the pain relief from the anesthetic and the inventive technique lasted more than 24 hours. When this anesthetic was sprayed onto the SPG nerve in the manner described previously, an extremely effective headache control was observed. Patients were returned to work and avoided other toxic pain medication almost 100% of the time.

Among the many advantages of the inventive apparatus and method are:
1. Outstanding performance.
2. Superb capabilities for controlling headaches.
3. Excellent ability to control facial pain.
4. Superior control and management of pain resulting from headaches.
5. Particularly useful for controlling headaches and facial aches and pain resulting therefrom for patients suffering from a condition of one or more of the following: sphenopalatine neuralgia, trigeminal neuralgia, glossopharynged neuralgia, migraine, migraine with aural headache, migraine without aura headache, tension headache, cluster headache, chronic cluster headache, paroxysmal hemicranias, superior laryngeal neuralgia, facial pain, atypical facial pain, or herpes zoster opthalmicus.
6. Patients can utilize the novel apparatus and technique safely and effectively at home.
7. Substantially reduces the discomfort and cost of treating headaches.
8. Recognizes that the correct three dimensional positioning of the SPG nerve in relation to the human nostril and is capable of accommodating small variations in terms of sizes among different people.
9. Can be utilized effectively clinically.
10. Can revolutionize treatment options.
11. Can help millions of people with recurring headaches live a productive and happy live and saves billions of dollars for U.S. alone.
12. Can be used by patients and medical personnel alike.
13. Can be used by patients independently and without the presence of a doctor or other medical specialist.
14. Reliable.
15. User friendly.
16. Easy to use.
17. Durable.
18. Economical.
19. Attractive.
20. Safe.
21. Efficient.
22. Effective.

Although embodiments and examples of the invention have been shown and described, it is to be understood that various modifications, substitutions, and rearrangements of parts, components, and/or process (method) steps, as well as other uses of the apparatus and/or method (technique), can be made by those skilled in the art without departing from the novel spirit and scope of this invention.

What is claimed is:

1. A headache controlling apparatus for controlling headaches and facial aches, comprising:
a slidable and controlling moveable injector with a passageway for passing, delivering and injecting an anesthetic, comprising
an elongated tubular section having a posterior portion comprising a rearward end and a anterior portion comprising a front end;
a container-supporting semi-rigid stem providing a superior pedestal extending outwardly from the posterior portion;
a nozzle extending forwardly from the anterior portion of the tubular section, said nozzle having a tip and defining an array of apertures for spraying an anesthetic toward a sphenopalatine ganglion (SPG nerve);
a container containing an anesthetic and secured to the outer end of the stem and communicating with the passageway in the injector for injecting the anesthetic through the passageway of the stem and tubular section through the apertures of the nozzle;
a nostril-engaging introducer having a narrow anterior section providing an underside with a general planar surface and a concave posterior section having a larger cross-sectional than said narrow anterior section, said concave posterior section having a contour generally complementary and conforming to the interior of a patient's nostril for insertion in the nostril, said introducer having a tube-receiving passageway extending therethrough for slidably receiving the tubular section of the injector; and
a manually grippable handle securely connected to a back portion of the introducer and defining an upwardly facing channel providing a track communicating with the tube-receiving passageway of the introducer for slidably receiving the elongated tubular section of the injector;
whereby when the handle is pushed towards the patient's face until the introducer snugly and comfortably engages and fits within the nostril of the patient to lift the flat tip of the nose, and thereafter the stem and nozzle are pushed toward the patient's nose to slide the elongated tubular section and nozzle rearwardly until the nozzle is located medially, posteriorly and inferiorly to the SPG so as to be positioned in proximity of and rearwardly and downwardly of the SPG nerve so that the anesthetic in the container can be dispensed to inject a spray of the anesthetic through the apertures of the nozzle about the SPG nerve to substantially control, decrease and minimize pain from a headache or facial ache.

2. A headache controlling apparatus in accordance with claim 1 wherein said headache treating apparatus is disposable.

3. A headache controlling apparatus in accordance with claim 1 wherein:
said injector comprises flexible plastic; and
said introducer is elastomeric and resilient.

4. A headache controlling apparatus in accordance with claim 1 wherein:
said injector is selected from the group consisting of a left nostril-engaging injector and a right nostril-engaging injector; and
said left nostril-engaging injector has a different nozzle that said right nostril-engaging injector.

5. A headache controlling apparatus in accordance with claim 1 wherein:
said introducer is selected from the group consisting of a left nostril-engaging introducer and a right nostril-engaging introducer; and
said left nostril-engaging introducer has a different and generally complementary contour to said right nostril-engaging introducer.

6. A headache controlling apparatus in accordance with claim 1 container is selected from the group consisting of: a squeezable container, a canister, a pressurized container, an aerosol can, and a syringe.

7. A headache controlling apparatus in accordance with claim 1 wherein said anesthetic composition comprises benzocaine, tetracaine and ropivacaine.

8. A headache controlling apparatus in accordance with claim 7 wherein the anesthetic comprises by weight based on the total weight of the anesthetic:
about 14% benzocaine;
about 2% tetracaine; and
about 1% ropivacaine.

9. A headache controlling apparatus in accordance with claim 1 wherein said nozzle extends in the lateral, anterior and superior direction at an angle of inclination ranging from about 45 degrees to about 60 degrees.

10. A headache controlling apparatus in accordance with claim 1 wherein:
the anterior section comprises a tip that extends from about 1 cm to about 3 cm; and
the concave posterior section extends from about 2 cm to about 3 cm.

11. A method for controlling headaches and facial aches, comprising the step of:
inserting a nozzle of an injector comprising a tubular section of an injector through a nostril;
positioning the nozzle at an upward angle of inclination in proximity and downwardly and rearwardly of a sphenopalatine ganglion (SPG nerve);
injecting an anesthetic through apertures of the nozzle upwardly, laterally and anteriorly and towards the SPG nerve; and
spraying the anesthetic about the SPG nerve to minimize pain and control a headache or facial ache.

12. A method for controlling headaches and facial aches in accordance with claim 11 wherein said anesthetic composition comprises benzocaine, tetracaine, and ropivacaine.

13. A method for controlling headaches and facial aches in accordance with claim 11 including:
pushing or placing an introducer snugly and comfortably within a nostril to lift the tip of the nose before positioning the nozzle in proximity to the SPG nerve; and
the nozzle is positioned at an upward angle of inclination in proximity and downwardly and rearwardly of a sphenopalatine ganglion (SPG nerve).

14. A method for controlling headaches and facial aches in accordance with claim 13 sliding the tubular section of the injector through a passageway in the introducer.

15. A method for controlling headaches and facial aches in accordance with claim 14 including pushing the introducer with a handle.

16. A method for controlling headaches and facial aches in accordance with claim 15 including sliding the tubular section of the introducer on a track of the handle.

17. A method for controlling headaches and facial aches in accordance with claim 11 including squeezing a container secured on the stem of the tubular section to inject and spray the anesthetic.

18. A method for controlling headaches and facial aches in accordance with claim 11 including controlling the pain in about 30 seconds to about 60 seconds after said injecting.

19. A method for controlling headaches and facial aches in accordance with claim 11 including controlling the pain and headache or facial ache for about 4 hours to about 24 hours after said injecting.

20. A method for controlling headaches and facial aches in accordance with claim 11 for patients suffering from a condition selected from the group consisting of: sphenopalatine neuralgia, trigeminal neuralgia, glossopharynged neuralgia, migraine, migraine with aural headache, migraine without aura headache, tension headache, cluster headache, chronic cluster headache, paroxysmal hemicranias, superior laryngeal neuralgia, facial pain, atypical facial pain, and herpes zoster opthalmicus.
